(19) United States
(12) Patent Application Publication
(10) Pub. No.: US 2003/0013124 A1
(43) Pub. Date: Jan. 16, 2003

(54) Surfaces for covalent attachment of ligands

(76) Inventors: Jennifer M. Lu Kuo, Bedford, MA (US); David Ho, Boxborough, MA (US); Tai-Nang Huang, Lexington, MA (US)

Correspondence Address: Y. ROCKY TSAO, Fish & Richardson P.C., 225 Franklin Street, Boston, MA, 02110-2804, US

(21) Appl. No.: 10/142,634

(22) Filed: May 10, 2002

Related U.S. Application Data
(60) Provisional application No. 60/293,888, filed on May 24, 2001.

(51) Int. Cl. G01N 33/53; C08F 265/10; C08F 267/10

(52) U.S. Cl. 435/7.1; 525/296

(57) ABSTRACT

This invention relates to a solid substrate that has a modified surface to which a sulfoamido group is attached via a linker.

### CROSS REFERENCE TO RELATED APPLICATION

Pursuant to 35 U.S.C. § 119(e), this application claims the benefit of prior U.S. provisional application No. 60/293,888, filed May 24, 2001.

### BACKGROUND

Many analytical and preparative methods in biology and chemistry require the attachment of target compounds, such as peptide ligands or oligonucleotide probes, to a solid support. Frequently, different species of target compounds are attached onto the surface of the solid support, each at a discrete location. Attachment can be achieved in a number of different ways, including covalent and non-covalent interaction. Typically, covalent attachment is more robust. See, for example, Lamture et al. (1994) Oligonucleotide Research 22: 2121-2125; Beattie et al. (1995) Mol. Biotechnol. 4: 213-225; Joos et al. (1997) Anal. Biochem 247: 96-101; Rogers et al. (1999) Anal. Biochem. 266: 23-30; and Chrisey et al. (1996) Oligonucleotide Research 24: 3031-3039.

Protocols have been developed to covalently attach a target compound to a support surface. One exemplary protocol includes synthesizing an oligonucleotide directly on a support surface using stepwise photolithographic reactions. Another exemplary protocol includes depositing a target nucleic acid, such as a cloned cDNA, a PCR product, or a synthetic oligonucleotide onto a surface of a solid support, e.g., a microscopic glass slide, in the form of an array. The surface can be modified in order to facilitate the attachment of the nucleic acid. The array is used in hybridization assays to determine the presence or abundance of particular sequences in a sample.

### SUMMARY

The invention is based, in part, on the discovery of a new method of modifing the surface of a solid substrate. The modified surface is useful, for example, for the covalent attachment of target compounds, such as oligonucleotides.

One aspect of this invention relates to a solid substrate that includes a chemical group covalently bonded to its surface. The chemical group is of formula (I) shown below:

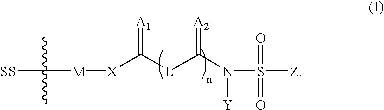

In the formula, SS is the surface; n is 0-8; X is a bond, O, S, or NH; Y is H, alkyl, arylalkyl, or heteroarylalkyl, wherein the alkyl, aralkyl, or heteroarylalkyl is optionally substituted with an electron-withdrawing group; Z is hydrogen, hydroxy, alkyl, alkenyl, alkynyl, aryl, or heteroaryl, wherein the alkyl, alkenyl, alkynel, aryl, or heteroaryl is optionally substituted with alkyl, halo, hydroxy, amino, carboxy, or oxo; each of A₁ and A₂, independently, is O, S, or NH; L is alkylene, alkenylene, or alkynylene, and is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and M is a bond or alkylene, alkenylene, or alkynylene, wherein the alkylene, alkenylene, or alkynylene is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—.

Embodiments of the above-described solid substrate include those in which n is 0-4; those in which X is NH; those in which Y is H or alkyl optionally substituted with an electron-withdrawing group; those in which Z is alkyl, aryl, or heteroaryl, optionally substituted with alkyl, halo, hydroxy, amino, carboxy, or oxo; those in which each of A₁ and A₂, independently, is O; those in which L is alkylene (e.g., ethylene); and those in which M is a bond.

The term “alkyl,” alone or in combination (e.g., as in heteroarylalkyl), refers to a C₁₋₁₀ straight or branched hydrocarbon chain, containing the indicated number of carbon atoms. The terms “alkenyl” and “alkynyl” respectively refer to a C₁₋₁₀ straight or branched hydrocarbon chain containing at least one double bond and a C₁₋₁₀ straight or branched hydrocarbon chain containing at least one triple bond. The term “alkylene” refers to a divalent alkyl group (i.e., —R—). Likewise, the term “alkenylene” and “alkynylene” respectively refer to a divalent C₁₋₁₀ alkenyl group and a divalent C₁₋₁₀ alkynyl group, respectively. The term “aryl” refers to a 6-carbon monocyclic or 10-carbon bicyclic aromatic ring system in which each ring may be mono-, di-, or multi-substituted. Examples of aryl groups include phenyl and naphthyl. The term “arylalkyl” refers to alkyl substituted with an aryl. The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms being O, N, or S. Each ring of the heteroaryl may be mono-, di-, or multi-substituted. Examples of heteroaryl groups include pyridyl, furyl or furanyl, imidazolyl, benzimidazolyl, pyrimidinyl, thiophenyl or thienyl, quinolinyl, indolyl, and thiazolyl. The term “electron-withdrawing group” refers to a functional group that draws electrons to itself more than a hydrogen atom would if it occupied the same position as the electron-withdrawing group in a molecule. Examples of electron-withdrawing groups include a positively charged group, halogen, cyano, nitro, carbonyl, carbamido, carbamyl, carboxyl, thioureido, thiocyanato, and sulfoamido. By “substituting” or “substitution,” it is meant that at least one or more substituents (which can be the same or different) can be attached to any moiety of the base group. For example, in “arylalkyl substituted with halo,” the halo substituent can be on either aryl or alkyl.

The term “solid substrate,” as used herein, includes both flexible and rigid substrates. By “flexible” is meant that the solid substrate is pliable. For example, a flexible substrate can be bent, folded, or similarly manipulated to at least some extent without breakage. The surface of a solid substrate may include a planar surface (e.g., a slide or a plate), convex surface (e.g., a bead), concave surface (e.g., a well), and so forth. Potentially useful solid substrates include: mass spectroscopy plates (e.g., for MALDI), glass (e.g., functionalized glass, a glass slide, porous silicate glass, a single crystal silicon, quartz, UV-transparent quartz glass), plastics and polymers (e.g., polystyrene, polypropylene, polyvinylidene difluoride, poly-tetrafluoroethylene, polycarbonate, PDMS, acrylic), metal coated substrates (e.g., gold), silicon substrates, latex, membranes (e.g., nitrocellulose, or nylon), and a refractive surface suitable for surface plasmon resonance. Solid substrates can also be porous. Useful porous substrates include: agarose gels, acrylamide gels, sintered glass, dextran, meshed polymers (e.g., macroporous crosslinked dextran, sephacryl, and sepharose), and so forth.

Other embodiments of the solid substrate include those in which n is 1; those in which Y is nitromethyl or cyanomethyl; those in which Z is (4-methyl)phenyl; those in which L is ethylene.

Another aspect of this invention relates to a method for preparing a solid substrate having a modified surface. The method includes providing a solid substrate that contains a SS—M—X—H group and reacting the solid substrate with a coupling compound of the following formula

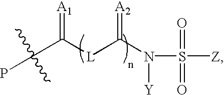

in which P is —OH, —NH₂, or —SH, to convert the surface to a chemical group of formula (I) shown above.

The invention also relates to a method for covalently attaching a target compound having a nucleophilic group. The method includes providing a solid substrate having a surface covalently bonded to a group of formula (I) shown above, and contacting a target compound to the surface, whereby the nucleophilic group reacts with the activated group to covalently bond the target compound to the surface. The target compound can be uniformly or differentially disposed on the surface, with a density of the compound on the surface of 0.1 to 10 pmol/cm². Examples of the target compound include polymeric compounds, such as an oligonucleotide, a peptide, a polypeptide, a polysaccharide, or a combination thereof; monomeric compounds, such as a nucleoside, an amino acid, or a monosaccharide; and other organic compounds, e.g., a non-polymeric compound have a molecular weight of at least 50, 100, 500, 1000, 5000, 10,000, or greater. The target compounds include analogs of naturally occurring compounds.

A “nucleophilic group” refers to a chemical moiety that is rich in electron and tends to react with electron-deficient moiety within a compound. Examples of a nucleophilic group include anions (e.g., HO⁻), alkoxy (e.g., —OCH₃), arylthio (e.g., —SC₆H₅), amino (e.g., —NH₂), and aryl (e.g., pyridinyl).

Also within the scope of this invention is a solid substrate which includes a modified surface with a plurality of addresses, wherein each address has attached thereto a compound of formula (I) shown above.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This invention relates to a solid substrate having a surface characterized by a covalently bonded activated group that includes an electron-withdrawing group on an N-substituted sulfonamide. The activated group can be used to attach a target compound, e.g., a target compound having an amino leaving group, to the surface.

The solid substrate can be prepared by a method known in the art. For instance, one can first provide a substrate having a reacting group SS—M—X—H (each of SS, M, and X is defined as above); attaching a molecule to the substrate to form a covalent bond between the reacting group and the molecule, thereby obtaining a substrate having a modified surface having a chemical group of formula (I).

Shown below is a scheme that depicts synthesis of a molecule (top) and modification of a surface to provide a highly activated substrate (bottom):

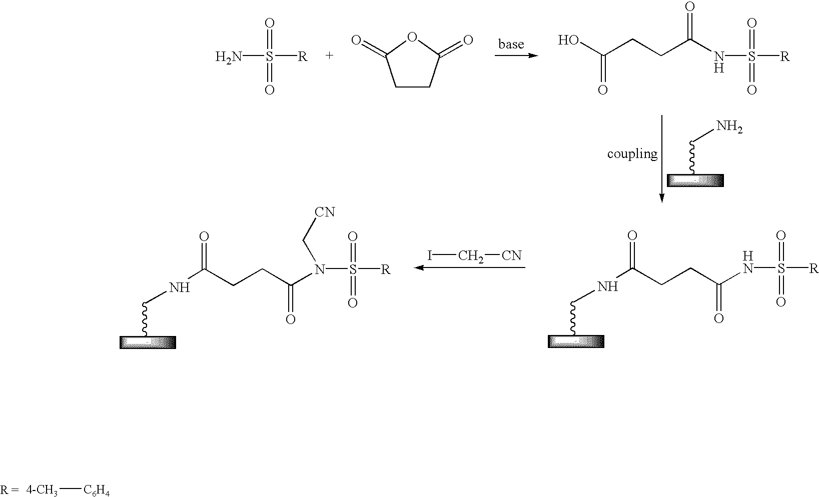

In this method, a sulfonamide is first coupled with an anhydride in the presence of a base, e.g., diisopropylethylamine, to produce a carbamide molecule that can be used to modify a surface of a solid substrate. The carbamide molecule is then coupled with an amino group on the surface to covalently bond the molecule to the surface. The modified surface is reacted with an iodoacetonitrile to produce a N-cyanomethyl-sulfonamide. Other compounds (e.g., those in which A₁ and A₂ are not both O, or n is not 1 or Z is alkyl) can be prepared by similar methods.

As shown below, the sulfoamide can undergo a reaction with a target molecule (e.g., a polynucleotide) so that the target molecule can be covalently bonded to the surface of a solid substrate.

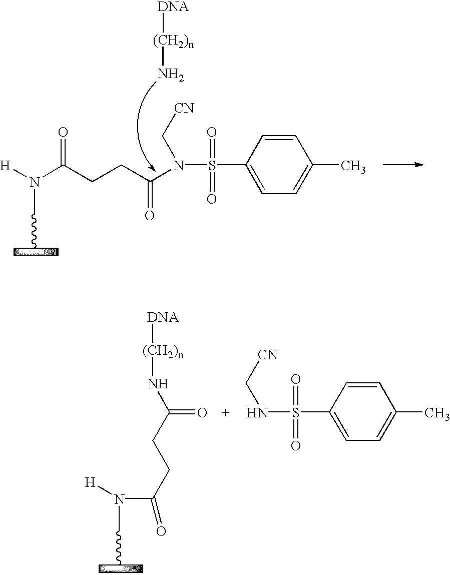

This reaction can occur in only a few minutes at ambient temperature and may be carried out in a variety of mediums such as water, an aqueous buffer, and an organic solvent.

The solid substrate can be a solid or porous solid support. In some implementations, the support is a bead, microparticle, a nanoparticle, a matrix, or a gel. Beads, microparticles, and nanoparticles can be used, e.g., in chemical and library screening applications. Beads, matrices, gels and other solid supports can be used, e.g., in ligand purification methods, e.g., as a matrix for column chromatography. The beads can include interior surfaces that increase effective surface area and also partition components. Some particles include a radiofrequency tag that can uniquely identify the particle (e.g., as described in U.S. Pat. No. 5,262,530).

The substrate used herein can be made from any material either flexible or rigid. In general, the substrate material is resistant to the variety of synthesis and analysis conditions of the combinatorial chemical assays. Examples of substrate materials include, but are not limited to, glass, quartz, silicon, gallium aresenide, polyurethanes, polyimides, and polycarbonates. Of course, the substrate material can be a composite of one or more materials. For example, glass supports, i.e., glass slides, can be coated with a polymer material to produce a substrate. Additionally, the support can be made in any shape, e.g., flat, tubular, round, and include etches, ridges or grids to create a patterned substrate. The substrate can be opaque, translucent, or transparent. The substrate can include wells or moats.

This invention also relates to a method for covalently attaching a target compound to a solid support. The target compound can be a polymeric compound or a monomeric compound. It can be prepared using any known methodology. The particular method for preparing a target compound, such as a modified target compound, to include the requisite reactive group will depend on the nature of the target compound and the nature of the reactive group, which is to be incorporated into the compound. For example, where the target compound is an oligonucleotide, a number of protocols exist for producing an oligonucleotide with or without a reactive group. For instance, an unmodified oligonucleotide can be synthesized on a DNA/RNA synthesizer using a standard phosphoramidite chemistry. A reactive group can be present on a modified phosphoramidite, which can be incorporated into any position of the oligonucleotide during synthesis. Alternatively, a reactive group can be enzymatically added to one of the termini of an oligonucleotide. In another example, where the target compound is a peptide, it can be prepared chemically (e.g., on a peptide synthesizer) or biologically (e.g., expressed from a host cell or in vitro translated). The moiety in peptide, such as carboxy, hydroxy, phenoxy, amino, guanidino, or thio, can serve as a reactive group. An additional reactive group can be introduced into a modified peptide by, for example, incorporation of a modified amino acid.

An example of the target compound is an oligonucleotide, which can be covalently attached to the solid substrate of this invention at either the 3′ or the 5′ terminus, or alternatively, at a specific position along the sequence. The oligonucleotide can be a synthetic DNA, a synthetic RNA, a cDNA, a mRNA, or a PNA, which is generally at least about 5, 10, or 15 nucleotides in length, and may be as long as 2000, 3000, or 5000 nucleotides or longer.

The oligonucleotide molecules can be attached to the solid substrate randomly, or in an order. Preferably, the oligonucleotide molecules are arranged into an ordered array. As used herein, an ordered array is a regular arrangement of molecules, as in a matrix of rows and columns. The methods of the present invention are such that an individual array can contain a number of unique attached oligonucleotide molecules. The array can contain more than one distinct attached oligonucleotide molecule.

Also within the scope of this invention is an array fabricated on a solid substrate of this invention. A target compound, such as an oligonucleotide, a peptide, a polysaccharide, a nucleoside, an amino acid, a monosaccharide, or another organic compound, can be deposited on the solid substrate in the form of an array. The array thus described can be used in a variety of applications. For example, the presence of a particular analyte in a given sample is detected qualitatively or quantitatively. More specifically, the sample suspected of containing the analyte of interest is contacted with the array under conditions sufficient for the analyte to interact with its respective pair member that is present on the array. Thus, if the analyte of interest is present in the sample, it can form a complex with its pair member on the array. The presence of the complex on the array can be detected by a detectable label such as an enzymatic, isotopic or fluorescent label. The detectable label can include a signal production system such as a chemiluminescent system or a proximity detection system.

The afore-mentioned array can have a density of at least 10, 50, 100, 200, 500, 10³, 10⁴, 10⁵, 10⁶, 10⁷, 10⁸, or 10⁹ addresses per cm², and/or a density of no more than 10, 50, 100, 200, 500, 10³, 10⁴, 10⁵, 10⁶, 10⁷, 10⁸, or 10⁹ addresses/cm². Preferably, the plurality of addresses includes at least 10, 100, 500, 1,000, 5,000, 10,000, or 50,000 addresses, or less than 9, 99, 499, 999, 4,999, 9,999, or 49,999 addresses. The center to center distance between addresses can be 5 cm, 1 cm, 100 mm, 10 mm, 1 mm, 10 nm, 1 nm, 0.1 nm or less and/or ranges between. The longest diameter of each address can be 5 cm, 1 cm, 100 mm, 10 mm, 1 mm, 10 nm, 1 nm, 0.1 nm or less and/or ranges between. Each address contains 10 mg, 1 mg, 100 ng, 1 ng, 100 pg, 10 pg, 0.1 pg, or less of a target compound and/or ranges between. Alternatively, each address contains 100, 10³, 10⁴, 10⁵, 10⁶, 10⁷, 10⁸, or 10⁹, or more molecules of the reactive compound attached thereto and/or ranges between. Addresses in addition to addresses of the plurality can be deposited on the array. The addresses can be distributed, on the substrate in one dimension, e.g., a linear array; in two dimensions, e.g., a planar array; or in three dimensions, e.g., a three dimensional array.

A substrate with a planar surface having the activated chemistry described herein can be used to generate an array of a diverse set of target compounds. In one exemplary application, oligonucleotide probes of differing sequence are positioned on the array surface. Such an oligonucleotide array can be used to query a complex sample and generate a large data set. This application and similar hybridization based applications can be used for gene discovery, differential gene expression analysis, sequencing, or genomic polymorphism analysis. Further, such oligonucleotide arrays are particularly amenable to high-throughput applications. Other exemplary applications are polypeptide arrays, e.g., arrays of antigens and/or antibodies.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications, including patents, cited herein are hereby incorporated by reference in their entirety.

### EXAMPLE 1

A. Generation of Amino Groups on Glass Slides:

25×75 mm glass slides (VWR International, West Chester, Pa., Cat. No. 48300-025) were washed thoroughly with de-ionized water, immersed in 6 N HCl overnight, and then washed with acetone. These glass slides were baked at 105° C. in an oven for 30 minutes. The slides were then coated with 5.6% gamma-amino propyltriethoxysilane (GAPS) in toluene at 80° C. for 16 hours. The coated slides were washed sequentially with toluene, methanol, and methanol/water solution. Un-reacted silanols on the surface were capped with 50% chlorotrimethylsilane solution in pyridine. The slides were washed with methanol and ethyl ether, and then blow-dried with nitrogen. These slides, “amino glass slides,” were modified with 3-carboxypropionyl-p-toluenesulfonamide as described in Example 1, section C, below.

B. Synthesis of 3-carboxypropionyl-p-toluenesulfonamide:

The compound 3-carboxypropionyl-p-toluenesulfonamide was synthesized by reacting p-toluenesulfonamide (8.62 g, 50.3 mmol) with succinic anhydride (6.12 g, 24.9 mmol) in the presence of diisopropylethylamine (21.5 mL, 123 mmol) and a catalytic amount of 4-dimethylaminopyridine (0.62 g, 5.1 mmol) in acetonitrile (85 mL) at room temperature overnight. The organic solvent of the reaction mixture was evaporated using a rotary evaporator. 75 mL of 1.0 N sodium hydroxide solution was added to the oily residue with mixing. The resulting solution was washed with 75 mL of methylene chloride. The aqueous solution was then acidified by addition of approximately 110 mL of 1.0 N hydrochloric acid. A white cloudy solution was observed. White crystals were collected and dried using a water aspirator and then under vacuum for overnight. The product was 3-carboxypropionyl-p-toluenesulfonaminde in the form of a white crystalline material (12.3 g, 90% yield). The structure of the product was confirmed by NMR analysis.

mp: 167-169° C.

¹H NMR (400 MHz, DMSO-d₆): δ 5 2.35 (t, 2H), 2.39 (s, 3H), 2.43 (t, 2H), 7.78 (d, 2H), 7.41 (d, 2H), and 12.1 (s, 2H).

C. Coating of 3-carboxypropionyl-p-toluenesulfonamide to Primary Amino Groups on Glass Slides:

A solution of 1.08 g of 3-carboxypropionyl-p-toluenesulfonamide (0.2 M) and 2.18 g of benzotriazo-1-yloxytripyrrolidinophosphonium hexafluorophosphate (PyBOP, 0.21 M) in 18.5 mL of N, N-dimethylformamide (DMF) was prepared. This solution was mixed with 1.5 mL of diisopropylethylamine in a slide cartridge. Amino glass slides were immersed into the cartridge. The reaction mixture with the slides was agitated with an orbital shaker on a magnetic stirrer plate overnight. The coated slides were washed with DMF, methylene chloride, and methanol. The washed slides were dried under vacuum.

D. Activation of the Coated Slides:

A mixture of 1.5 mL of diisopropylethylamine, 2.2 mL of iodoacetonitrile, and 16 mL of N-methyl-2-pyrrolidinone (NMP) was prepared in a slide cartridge. The coated slides prepared in Example 1, Section C (above), were immersed in the mixture in a dry box. The reaction mixture was agitated using an orbital shaker on a magnetic stirrer plate for ˜24 hr at room temperature. The slides were washed with NMP and methylene chloride and were dried under vacuum. These slides, the “activated slides,” were stored and later used as described in the Examples below.

### EXAMPLE 2

A. Covalent Attachment of an Oligonucleotide at Different Concentrations:

A 5′-Cy3-labeled and 3′-amino-modified oligonucleotide probe of 21 nucleotides in length (5′-Cy3-GTACTGCACCAGGCGGCCGCA-NH₂-3′, SEQ ID NO: 1) was spotted onto the activated slides from Example 1 (Section D). The probe was tested at a variety of concentrations, 1.25, 2.5, 5, 10, 20, and 40 μM, in each of three different spotting solutions: 150 mM sodium phosphate, pH 8.5; 50% Micro Spotting Solution (TeleChem, Sunnyvale Calif.; and 3×SSC. The solutions were spotted onto the activated slides using a 0.787 mm solid pin (V&P Scientific) that produces a 35 nL hanging drop. After spotting, the slides were dried overnight at room temperature and then scanned at 30 μm resolution in a ScanArray 4000 (Packard Biochip, Meriden Conn.) with a laser power setting of 70 and PMT gain of 70. Then, the slides were blocked for 2 h at room temperature using NoAb® 1×Pre-Hybridization/Blocking Buffer (NoAb Diagnostics, Ontario Canada) containing primary and secondary amino groups. After blocking, the slides were washed twice with 0.1×SSC/0.1% SDS, rinsed briefly in 0.1×SSC and H₂O. The slides were scanned again at a laser power setting of 70 and PMT gain of 70.

Prior to blocking and washing steps, the fluorescence signal intensities for the Cy3-labeled oligonucleotides at spotting concentrations ranging from 1.25 μM to 40 μM were similar for all slides. Signal intensities were reduced after blocking and washing steps. The fluorescence signal intensity of each spot on the various slides was analyzed using QuantArray 2.0 (Packard Biochip, Meriden Conn.) software. The net fluorescence signal intensity was calculated by subtracting the average background signal from the signal measured at each spot. With all three spotting solutions, 150 mM sodium phosphate, pH 8.5, 50% Mirco Spotting solution (TeleChem, Sunnyvale Calif.), and 3×SSC, a concentration-dependent increase in net fluorescence signal intensities was observed.

Two reference slides that have a different surface chemistry from the activated slides described above were treated in parallel to compare performance. The glass slides treated with aldehyde compounds such as the aldehyde slides (ALS-25) from CEL ASSOCIATES (Houston, Tex.) were used as the reference slides. The spotted probe also bound to the reference slides in a concentration dependent manner regardless of the spotting solution used.

However, compared to the two reference slides, the slides described herein exhibited stronger fluorescence signal intensities for all three spotting solutions at each of the concentrations tested. Curves were generated for net fluorescence intensity versus concentration of probe in the spotting solution. The slope of the curves was steepest for the activated slides of Example 1 compared to the two reference slides. In addition, the activated slides were efficiently saturated with a solution of spotting oligonucleotide. These unexpected results are indicative of the high performance properties of the reactive group on the slides of Example 1.

B. Kinetics of Covalent Attachment

The 5′-Cy3-labeled and 3′-amino-modified oligonucleotide probe just described was prepared at a concentration of 1.25, 2.5, 5, 10, 20, and 40 μM in 150 mM sodium phosphate and spotted in duplicate in reverse chronological order at 18, 4, 2, 1, 0.5, and 0.25 hours onto activated slides from Example 1. At time 0, the slides were scanned at 30 μm resolution in a ScanArray 4000 (Packard Biochip, Meriden Conn.) with a laser setting of 70 and PMT gain of 70. Then, the slides were blocked for 2 hours at room temperature using NoAb® 1×Pre-Hybridization/Blocking Buffer (NoAb Diagnostics, Ontario Canada), washed twice with 0.1×SSC/0.1% SDS, and rinsed briefly in 0.1×SSC and H₂O. The slides were scanned first with a laser setting of 70 and PMT gain of 70 and then with a laser setting of 70 and PMT gain of 60. Finally, the slides were sonicated for 30 minutes at room temperature in 0.1×SSC/0.1% SDS, rinsed briefly in 0.1×SSC and H₂O, and scanned again with a laser setting of 70 and PMT gain of 60.

The kinetics of covalent attachment of the Cy3-labeled, amino-modified oligonucleotides to activated slides of the invention was compared to that of the reference slides. Both types of slides exhibit similar fluorescence intensities before blocking and washing. Scans taken after blocking and washing, at a laser setting of 70 and PMT gains of 70 and 60, shows that the slides exhibit faster kinetics and higher attachment efficiency at all dispensing concentrations. Scans taken after sonication at laser setting of 70 and PMT gain of 60 indicate that the attachment of the spotted oligonucleotides remains permanently bonded on both slides.

Quantitative measurements of the kinetics of attachment of amine-modified oligonucleotide were obtained for both the activated slides of Example 1 and the reference slides as described above. The commercial slides exhibit a time-dependent increase in net fluorescence signal intensity, reaching a maximum of ˜4500 net fluorescence signal intensity with 40 μM spotted oligonucleotide after 18 hours of coupling. The slides also exhibit a time-dependent increase in net fluorescence signal intensity. The net fluorescence signal intensity generated using a spotting oligonucleotide concentration of 1.25 μM was more than twice of that of the intensity of spots on the references slides that were formed using the highest concentration of spotted oligonucleotide for the longest treatment time. Not only was the kinetics of the attachment of the spotted probe to the activated slides of example 1 much faster than one of the reference slides for 40 μM spotted oligonucleotide, but also the signal at saturation for the activated slide of Example 1 was at least twelve times stronger than that of the reference slide.

### EXAMPLE 3

An oligonucleotide array was fabricated and used for hybridization of Cy3-labeled complementary oligonucleotide samples. An oligonucleotide probe of 21 nucleotides in length (5′-NH₂-GTACTGCACCAGGCGGCCGCA-3′; SEQ ID NO: 2) was spotted onto the slides from Example 1 as described above. The probe was at a concentration of 25, 50, 100 and 200 μM in four different spotting solutions, namely, 50% DMSO, 50% Micro Spotting Solution, H₂O; or 3×SSC. After spotting, the slides were dried overnight at room temperature.

The slides were hybridized with 80 μL of 200 nM synthetic Cy3-labeled oligonucleotide target 5′-Cy3-TGCGGCCGCCTGGTGCAGTAC-3′ (SEQ ID NO: 3) in a Coverwell Perfusion Chamber (Grace Bio-Labs, Bend Oreg.) in a hybridization solution of 100 mM (2-[N-Morpholino]ethenesulphonic acid (MES), 1 M NaCl, 20 mM EDTA, and 0.01% (vol/vol) Tween 20. The hybridization was carried out overnight at room temperature in a humid plastic container. The slides were washed twice for 5 min each with 5×SSPE (0.75M NaCl, 50 mM NaH₂PO₄, 5 mM EDTA, pH7.4) and 0.01% (vol/vol) Tween 20, rinsed in H₂O, and scanned at 30 μm resolution in a ScanArray 4000 (Packard Biochip) with a laser setting of 70 and PMT gain of 70. Comparing to the reference slides, the slides from Example 1 exhibited the saturation of the fluorescence signal intensities at the lowest concentration of amino-modified oligonucleotide in all 4 spotting solutions, which suggested better attachment efficiency in the slides from Example 1.

The fluorescence signal intensities of the hybridized probe approached saturation more rapidly for the slides of Example 1 than the reference slides, regardless of the spotting solution used. This finding suggests that the attachment efficiency of the spotted oligonucleotide on the slides of Example 1 exceeds that of the reference slides, as measured by amount of hybridizable probe.

### OTHER EMBODIMENTS

From the above description, one skilled in the art can easily ascertain the essential characteristics of the present invention, and without departing from the spirit and scope thereof, can make various changes and modifications of the invention to adapt it to various usages and conditions. Accordingly, other embodiments are also within the scope of the following claims.

What is claimed is

1. A solid substrate comprising:
a surface; and
a chemical group covalently bonded to the surface, the chemical group having formula (I): 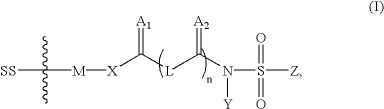
wherein
SS is the surface;
X is a bond, O, S, or NH;
Y is H, alkyl, arylalkyl, or heteroarylalkyl, wherein the alkyl, arylalkyl, or heteroarylalkyl is optionally substituted with an electron-withdrawing group;
Z is hydrogen, hydroxy, alkyl, alkenyl, alkynyl, aryl, or heteroaryl, wherein the alkyl, alkenyl, alkynel, aryl, or heteroaryl is optionally substituted with alkyl, halo, hydroxy, amino, carboxy, or oxo;
each of A₁ and A₂, independently, is O, S, or NH;
L is alkylene, alkenylene, or alkynylene, and is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—;
M is a bond or alkylene, alkenylene, or alkynylene, wherein the alkylene, alkenylene, or alkynylene is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and
n is 0-8.

2. The solid substrate of claim 1, wherein n is 0-4.

3. The solid substrate of claim 1, wherein n is 1.

4. The solid substrate of claim 3, wherein X is NH.

5. The solid substrate of claim 4, wherein Y is H or alkyl optionally substituted with an electron-withdrawing group.

6. The solid substrate of claim 4, wherein Z is alkyl, aryl, or heteroaryl, optionally substituted with alkyl, halo, hydroxy, amino, carboxy, or oxo.

7. The solid substrate of claim 4, wherein each of A₁ and A₂, independently, is O.

8. The solid substrate of claim 4, wherein L is ethylene and M is a bond.

9. The solid substrate of claim 5, wherein Y is alkyl optionally substituted with an electron-withdrawing group.

10. The solid substrate of claim 9, wherein Z is p-methylphenyl.

11. The solid substrate of claim 10, wherein each of A₁ and A₂, independently, is O.

12. The solid substrate of claim 10, wherein Y is —CH₂F, —CH₂Cl, —CH₂CN, or —CH₂NO₂.

13. The solid substrate of claim 12, wherein each of A₁ and A₂, independently, is O.

14. The solid substrate of claim 3, wherein Y is H or alkyl optionally substituted with an electron-withdrawing group.

15. The solid substrate of claim 14, wherein Z is alkyl, aryl, or heteroaryl, optionally substituted with alkyl, halo, hydroxy, amino, carboxy, or oxo.

16. The solid substrate of claim 14, wherein each of A₁ and A₂, independently, is O.

17. The solid substrate of claim 3, wherein Z is alkyl, aryl, or heteroaryl, optionally substituted with alkyl, halo, hydroxy, amino, carboxy, or oxo.

18. The solid substrate of claim 3, wherein each of A₁ and A₂, independently, is O.

19. The solid substrate of claim 1, wherein X is NH.

20. The solid substrate of claim 19, wherein Y is H or alkyl optionally substituted with an electron-withdrawing group.

21. The solid substrate of claim 19, wherein Z is alkyl, aryl, or heteroaryl, optionally substituted with alkyl, halo, hydroxy, amino, carboxy, or oxo.

22. The solid substrate of claim 19, wherein each of A₁ and A₂, independently, is O.

23. The solid substrate of claim 19, wherein L is alkylene.

24. The solid substrate of claim 1, wherein M is a bond.

25. The solid substrate of claim 1, wherein Y is H or alkyl optionally substituted with an electron-withdrawing group.

26. The solid substrate of claim 1, wherein Y is —CH₂F, —CH₂Cl, —CH₂CN, or —CH₂NO₂.

27. The solid substrate of claim 1, wherein Z is alkyl, aryl, or heteroaryl, optionally substituted with alkyl, halo, hydroxy, amino, carboxy, or oxo.

28. The solid substrate of claim 1, wherein each of A₁ and A₂, independently, is O.

29. The solid substrate of claim 1, wherein L is alkylene.

30. The solid substrate of claim 1, wherein M is a bond.

31. A method for preparing a solid substrate having a modified surface, comprising
providing a solid substrate having an SS—M—X—H group, wherein SS is a surface; X is a bond, O, S, or NH; and M is a bond or alkylene, alkenylene, or alkynylene, wherein the alkylene, alkenylene, or alkynylene is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and
reacting the solid substrate with a coupling compound containing the following chemical moiety 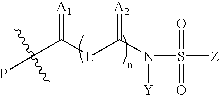
to convert the SS—M—X—H group to 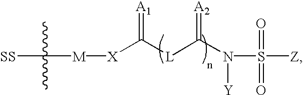
wherein
P is —OH, —NH₂, or —SH
Y is H, alkyl, arylalkyl, or heteroarylalkyl, wherein the alkyl, aralkyl, or heteroarylalkyl is optionally substituted with an electron-withdrawing group;
Z is hydrogen, hydroxy, alkyl, alkenyl, alkynyl, aryl, or heteroaryl, wherein the alkyl, alkenyl, alkynel, aryl, or heteroaryl is optionally substituted with alkyl, halo, hydroxy, amino, carboxy, or oxo;
each of A₁ and A₂, independently, is O, S, or NH;
L is alkylene, alkenylene, or alkynylene, and is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and
n is 0-8.

32. The method of claim 31, wherein P is hydroxyl.

33. The method of claim 32, further comprising contacting the surface with an alkyl halide, in which the alkyl is substituted with an electron-withdrawing group, when Y is not substituted with an electron-withdrawing group.

34. The method of claim 32, wherein L is alkylene and M is a bond.

35. The method of claim 32, wherein n is 1.

36. The method of claim 32, wherein X is NH.

37. The method of claim 32, wherein Y is CH₂NO₂ or CH₂CN.

38. The method of claim 32, wherein Z is alkyl, aryl, or heteroaryl, optionally substituted with alkyl, halo, hydroxy, amino, carboxy, or oxo.

39. The method of claim 32, wherein each of A₁ and A₂, independently, is O.

40. A method for covalently attaching a target compound having a nucleophilic group to a solid substrate, comprising:
providing a solid substrate having a group of formula (I): 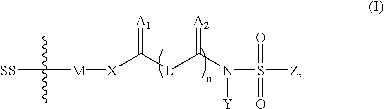
wherein
SS is a surface of the solid substrate;
X is a bond, O, S, or NH;
Y is H, alkyl, arylalkyl, or heteroarylalkyl, wherein the alkyl, aralkyl, or heteroarylalkyl is optionally substituted with an electron-withdrawing group;
Z is hydrogen, hydroxy, alkyl, alkenyl, alkynyl, aryl, or heteroaryl, wherein the alkyl, alkenyl, alkynel, aryl, or heteroaryl is optionally substituted with alkyl, halo, hydroxy, amino, carboxy, or oxo;
each of A₁ and A₂, independently, is O, S, or NH;
L is alkylene, alkenylene, or alkynylene, and is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—;
M is a bond or alkylene, alkenylene, or alkynylene, wherein the alkylene, alkenylene, or alkynylene is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and
n is 0-8; and
contacting a target compound containing a nucleophilic group with the surface of the solid substrate, whereby the nucleophilic group replaces the sulfonamide group in the solid substrate.

41. The method of claim 40, wherein the target compound is a nucleoside or a nucleic acid.

42. The method of claim 40, wherein the nucleophilic group is amino.

43. The method of claim 40, wherein L is alkylene and M is a bond.

44. The method of claim 40, wherein n is 1.

45. The method of claim 40, wherein Y is CH₂NO₂ or CH₂CN.

46. The method of claim 40, wherein X is NH.

47. The method of claim 40, wherein Z is alkyl, aryl, or heteroaryl, optionally substituted with alkyl, halo, hydroxy, amino, carboxy, or oxo.

48. The method of claim 40, wherein each of A₁ and A₂, independently, is O.

49. A solid substrate comprising a modified planar surface with a plurality of addresses, wherein each address has attached thereto a compound of formula (I): 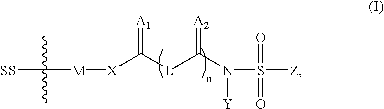
in which
SS is the surface of the solid substrate;
X is a bond, O, S, or NH;
Y is H, alkyl, arylalkyl, or heteroarylalkyl, wherein the alkyl, aralkyl, or heteroarylalkyl is optionally substituted with an electron-withdrawing group;
Z is hydrogen, hydroxy, alkyl, alkenyl, alkynyl, aryl, or heteroaryl, wherein the alkyl, alkenyl, alkynel, aryl, or heteroaryl is optionally substituted with alkyl, halo, hydroxy, amino, carboxy, or oxo;
each of A₁ and A₂, independently, is O, S, or NH;
L is alkylene, alkenylene, or alkynylene, and is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—;
M is a bond or alkylene, alkenylene, or alkynylene, wherein the alkylene, alkenylene, or alkynylene is optionally substituted with halo, hydroxy, nitro, amino, carboxyl, or oxo, or is optionally inserted with —O—, —CO—O—, —CO—NH—, —CO—N(alkyl)—, —NH—CO—, or —N(alkyl)—CO—; and
n is 0-8.
